(19) United States
(12) Patent Application Publication
(10) Pub. No.: US 2025/0354973 A1
(43) Pub. Date: Nov. 20, 2025

(54) SYSTEMS AND METHODS FOR INFILTROMETER TESTING OF SOIL BELOW GRADE

(71) Applicant: UPSTREAM TECHNOLOGIES, INC., FRIDLEY, MN (US)

(72) Inventor: RICHARD A KUNTZ, BIG LAKE, MN (US)

(21) Appl. No.: 18/663,825

(22) Filed: May 14, 2024

(51) Int. Cl. G01N 33/24

(52) U.S. Cl. CPC G01N 33/246

(57) ABSTRACT

A system for falling head infiltrometer testing includes an infiltrometer defining an infiltrometer passage within containing water. A first infiltrometer end of the infiltrometer is inserted into soil at a borehole bottom of a borehole to an insertion depth. A drill stem coupled to the second infiltrometer end of the infiltrometer traverses the infiltrometer within the borehole including insertion of the first infiltrometer end. A valve disposed proximate the first infiltrometer end is positionable to release the water from the infiltrometer passage into the soil surface of the soil at the borehole bottom of the borehole following insertion. A level detector detects a water surface level within the infiltrometer passage as a function of time as the water is absorbed into the soil following release. Data comprising the water surface level as a function of time may then be used to determine soil properties of the soil.

### CROSS-REFERENCE TO RELATED APPLICATIONS

U.S. Pat. No. 10,739,242 issued 11 Aug. 2020 and U.S. Pat. No. 11,353,391 issued 7 Jun. 2022 are hereby incorporated by reference in their entireties herein.

### BACKGROUND OF THE INVENTION

### Field

The present disclosure relates to measurement of soil properties by infiltrometer, and, in particular, to systems for falling head infiltrometer testing of soil below grade.

### Background

A falling head infiltrometer device may be used to measure the rate of water infiltration into soils. Various soil properties of the soil may then be determined from the rate at which water infiltrates into the soil as measured using the falling head infiltrometer device. The falling head infiltrometer device may be formed, for example, as a single ring that defines an infiltrometer passage. An end of the falling head infiltrometer device is inserted into the soil, and water is then added into the infiltrometer passage. Following the addition of the water, the decrease of the water surface of the water within the infiltrometer passage with respect to time as water infiltrates into the soil (i.e., the falling head) is observed and recorded. The observed decrease of the water surface with respect to time may then be used to determine soil properties of the soil. Note that the infiltrometer passage in such falling head infiltrometer devices is open to the atmosphere so that the air pressure above the water surface is atmospheric and a hydrostatic pressure distribution exists between the water surface and the soil surface.

Soil, as used herein, includes soil as well as other porous media. Soil properties, as used herein, may include, for example, porosity, sorptivity, hydraulic conductivity, and intrinsic permeability. Soil properties may include, for example, parameters used in various infiltration models such as, for example, the Lewis equation, Horton's equation, Phillip's equation, Green-Ampt model, Philip Dunne equation, and modified Philip Dunne equation (MPD). Conversely, these various infiltration models may be used to determine soil properties from the observed decrease of the water surface with respect to time within the infiltrometer passage. For example, see F. AHMED ET AL., A Modified Philip-Dunne Infiltrometer for Measuring the Field-Saturated Hydraulic Conductivity of Surface Soil, Vadose Zone J., Soil Science Society of America, Oct. 14, 2014, and also see ASTM Standard D8152-18 Standard Practice for Measuring Field Infiltration Rate and Calculating Field Hydraulic Conductivity Using the Modified Philip Dunne Infiltrometer Test, both of which are hereby incorporated by reference in their entireties herein.

It is sometimes desirable to determine these soil properties at some test elevation that may be at a depth below grade of several feet or more, not just at or nearly at the soil surface. Soil properties at test elevations below grade, for example, may be used for engineering foundations and pilings, earthwork engineering, engineering various subterranean structures, geotechnical exploration, and estimating transport of various material within the soil throughout the soil.

For example, in order to determine soil properties below grade (e.g., at some test elevation below grade within the soil), the soil is excavated to expose the soil at the test elevation at the location at which the soil properties are to be determined. The falling head infiltrometer device is then applied to the exposed soil at the location and test elevation to measure the decrease of the water surface with respect to time, which is then used to determine the soil properties. Such excavation may require an excavator or similar, a several man crew, shoring around the excavation to prevent collapse, and time. The resulting excavation may have the form of a pit, cavity, or scooped out area in the ground. Once the excavation is completed to expose the soil at the test elevation, additional time, manpower, materials, and equipment may be required to stabilize the excavation and to establish suitable conditions for conducting a falling head infiltrometer test. Weather such as rain, snow, below freezing conditions may interrupt, and, thus, prolong the infiltrometer testing process. Time is money, and the cost of the crew and the equipment over a several-day time period plus materials may be non-trivial. Thus, determining soil properties below grade by infiltrometer testing may be expensive, time consuming, and generally burdensome.

Accordingly, there is a need for improved systems for infiltrometer testing as well as related methods of use for the determination of soil properties of soil below grade.

### BRIEF SUMMARY OF THE INVENTION

These and other needs and disadvantages may be overcome by the systems for falling head infiltrometer testing and related methods disclosed herein. Additional improvements and advantages may be recognized by those of ordinary skill in the art upon study of the present disclosure.

In various aspects, the systems for falling head infiltrometer testing include an infiltrometer defining an infiltrometer passage within that may contain water and having a first infiltrometer end and a second infiltrometer end. The first infiltrometer end is configured for insertion into a soil surface of a soil at a borehole bottom of a borehole to an insertion depth within the soil, in various aspects. A drill stem coupled to the second infiltrometer end of the infiltrometer traverses the infiltrometer within the borehole to insert the first infiltrometer end into the soil at the borehole bottom and to withdraw the infiltrometer from the borehole, in various aspects. A valve is disposed proximate the first infiltrometer end of the infiltrometer, and the valve is positionable between a first valve position that retains the water within the infiltrometer passage, for example, during traversal of the infiltrometer within the borehole, and a second valve position that releases the water from the infiltrometer passage into the soil surface of the soil at the borehole bottom of the borehole following insertion of the first infiltrometer end into the soil to the insertion depth, in various aspects. A level detector may be placed within the infiltrometer passage, and the level detector is operable to detect a water surface level of a water surface of the water within the infiltrometer passage as a function of time and operable to generate data indicative of the water surface level as the function of time following release of the water from the infiltrometer passage, in various aspects. In various aspects, an internal video camera may be disposed within the infiltrometer passage in order to view the water surface within the infiltrometer passage. In various aspects, an external video camera may be attached externally to the infiltrometer to view insertion of the infiltrometer first end of the infiltrometer into the soil to the insertion depth. In various aspects, a tool may be provided to remove soil from the borehole bottom of the borehole. The tool may include a shaft configurable to extend at least a length of the borehole and a blade affixed in spiral shaped disposition to the shaft proximate a shaft end of the shaft and having a diameter generally commensurate with a diameter of a borehole passage. A blade end of the blade disposed nearest the shaft end is formed as an edge to scoop soil proximate a soil surface onto a surface of the blade by rotation of the blade resulting from rotation of the shaft, and a blade end of the blade disposed furthest from the shaft end is formed as a flange to retain soil upon the surface of the blade, in various aspects.

Related methods of use of the system for falling head infiltrometer testing are disclosed herein. In various aspects, the methods include the step of forming a borehole with a borehole bottom at a test elevation thereby exposing a soil surface of a soil at the borehole bottom of the borehole, and the step of inserting an infiltrometer first end of an infiltrometer into the soil to an insertion depth at the borehole bottom of the borehole using the drill stem coupled to a second infiltrometer end of the infiltrometer, the infiltrometer containing water within an infiltrometer passage. The methods may include the step of releasing the water from the infiltrometer passage into the soil surface of the soil at the borehole bottom of the borehole by positioning a valve from a first valve position retaining the water within the infiltrometer passage to a second valve position, the infiltrometer first end having been inserted into the soil to the insertion depth, in various aspects. The methods may include the step of collecting data indicative of changes of a water surface level of water within the infiltrometer passage as a function of time following the step of releasing the water from the infiltrometer passage into the soil surface, in various aspects.

This summary is presented to provide a basic understanding of some aspects of the apparatus and methods disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1 illustrates by a cut-away elevation view an exemplary implementation of a system for falling head infiltrometer testing according to the present inventions;

FIG. 2 illustrates by exploded perspective view the exemplary implementation of a system for falling head infiltrometer testing of FIG. 1;

FIG. 3 illustrates by cut-away elevation view portions of the exemplary implementation of a system for falling head infiltrometer testing of FIG. 1;

FIG. 4A illustrates by cut-away elevation view portions of the exemplary implementation of a system for falling head infiltrometer testing of FIG. 1;

FIG. 4B illustrates by cut-away elevation view portions of the exemplary implementation of a system for falling head infiltrometer testing of FIG. 1;

FIG. 5 illustrates by schematic diagram portions of the exemplary implementation of a system for falling head infiltrometer testing of FIG. 1;

FIG. 6A illustrates by perspective view portions of the exemplary implementation of a system for falling head infiltrometer testing of FIG. 1;

FIG. 6B illustrates by perspective view portions of the exemplary implementation of a system for falling head infiltrometer testing of FIG. 1;

FIG. 6C illustrates by cut-away elevation view certain aspects of the exemplary implementation of a system for falling head infiltrometer testing of FIG. 1;

FIG. 7 illustrates by schematic diagram portions of another exemplary implementation of a system for falling head infiltrometer testing;

FIG. 8 illustrates by process flow chart an exemplary method of operation of the exemplary implementation of a system for falling head infiltrometer testing of FIGS. 1, 7; and,

FIG. 9 illustrates by Cartesian plot exemplary data collected using the exemplary implementation of a system for falling head infiltrometer testing of FIGS. 1, 7.

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the implementations shown in the drawings and are utilized to facilitate description thereof. Use herein of relative terms such as generally, about, approximately, essentially, may be indicative of engineering, manufacturing, or scientific tolerances such as ±0.1%, ±1%, ±2.5%, ±5%, or other such tolerances, as would be recognized by those of ordinary skill in the art upon study of this disclosure.

### DETAILED DESCRIPTION OF THE INVENTION

A system for falling head infiltrometer testing is disclosed herein along with related methods of use. In various aspects, the system for falling head infiltrometer testing is configured to conduct a falling head infiltrometer test of soil exposed at a borehole bottom of a borehole. The borehole may be formed as a narrow shaft bored into the ground, and the borehole may be cased or uncased, in various aspects. The borehole may be bored in various ways to a test elevation at a location at which it is desirous to obtain soil property (ies), and the falling head infiltrometer test is then conducted of the soil at the borehole bottom. In certain aspects, the borehole may be formed using a hollow stem auger that then forms the casing around the borehole. The infiltrometer may be traversed through the casing, when present, in order to conduct the falling head infiltrometer test of the soil at the borehole bottom. By conducting the falling head infiltrometer test of the soil at the borehole bottom, the need for possibly extensive excavation required to expose the soil at the test elevation and test location may be eliminated.

In various aspects, the system for falling head infiltrometer testing includes an infiltrometer configured for falling head infiltrometer testing of the soil at the borehole bottom. The infiltrometer defines an infiltrometer passage within, and the infiltrometer has a first infiltrometer end and a second infiltrometer end. The infiltrometer passage contains water that is released into a soil surface of a soil at the borehole bottom of the borehole through the first infiltrometer end following insertion of the first infiltrometer end into the soil at the borehole bottom to an insertion depth, in various aspects. The decline in water level within the infiltrometer passage as a function of time is then detected. The detected water surface level z as a function of time t, for example, water surface levels z₁, z₂, z₃ . . . at corresponding times t₁, t₂, t₃ is then used to determine soil properties of the soil exposed at the borehole bottom according to a falling head infiltrometer test, in various aspects.

In various aspects, a drill stem is coupled to the second infiltrometer end of the infiltrometer to traverse the infiltrometer within the borehole including insertion of the first infiltrometer end into the soil at the borehole bottom to the insertion depth. In various aspects, a valve is disposed proximate the first infiltrometer end of the infiltrometer to control the release of water into the soil from the infiltrometer. The valve may be positioned between a first valve position that retains the water within the infiltrometer passage during traversal of the infiltrometer through the borehole and a second valve position that releases the water from the infiltrometer passage into the soil following insertion of the first infiltrometer end into the soil at the borehole bottom to the insertion depth, for example, by contact with the soil or electromechanically using a solenoid. In various aspects, a level detector may be placed within the infiltrometer passage that is operable to detect the water surface level of the water surface of water within the infiltrometer passage as a function of time and operable to generate data indicative of the water surface level as the function of time. In various aspects, an external video camera may be provided exteriorly of the infiltrometer to guide the infiltrometer into position including insertion into the soil to the insertion depth. In various aspects, an internal video camera may be provided within the infiltrometer passage to observe the water surface within the infiltrometer passage, for example, during the falling head infiltrometer test. In various aspects, a tool may be provided that is configured to remove soil proximate the borehole bottom in order to expose the soil surface that is to be tested. In various aspects, the plurality of water surface levels at the corresponding plurality of times is communicated to a computer, and the computer is configured to use the plurality of water surface levels at the corresponding plurality of times to determine the soil property. In various aspects, the soil property may include, for example, hydraulic conductivity, porosity, sorptivity, or intrinsic permeability. Various communication pathway(s) may be provided about the system for falling head infiltrometer testing to communicate variously, for example, analog signals, digital data, and electrical power.

As used herein, a computer includes a one or more processors, and may, in various aspects, include memory, display, microphone, speaker, mouse, keyboard, storage device(s), I/O devices, network interface, and so forth. Computer may include, for example, single-processor or multiprocessor computers, minicomputers, mainframe computers, as well as personal computers, hand-held computing devices, mobile devices, cellular telephones, tablets, watches, and other processor-based devices and combinations of processor-based devices, as would be readily recognized by those of ordinary skill in the art upon study of this disclosure. Computer may include one or more processors or processes distributed in a network cloud, in various aspects.

Apparatus, related methods of use, and related compositions of matter disclosed herein may be implemented, at least in part, in software having the form of computer readable instructions operably received by one or more computers to cause, at least in part, the one or more computers to function as at least a portion of the apparatus or to implement at least some of the steps of the methods of use. The methods of use disclosed herein may be implemented, at least in part, as a combination of hardware and operatively received software, in various aspects. Compositions of matter disclosed herein include non-transient computer readable media comprising computer readable instructions, the computer readable media being operably received by the one or more computers to cause the one or more computers, at least in part, to function as at least portions of the apparatus or to implement, at least in part, steps of the methods of use.

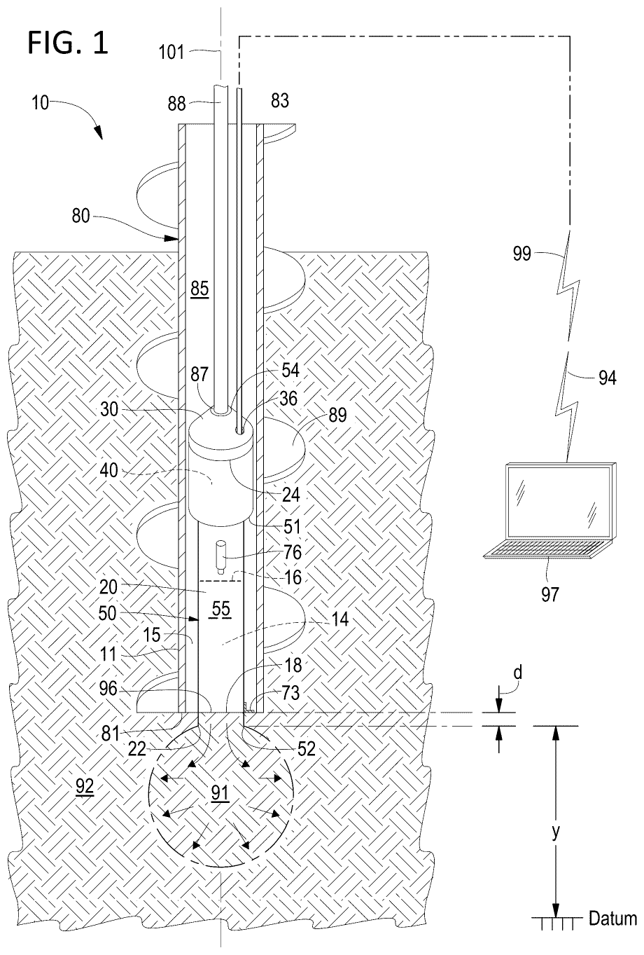

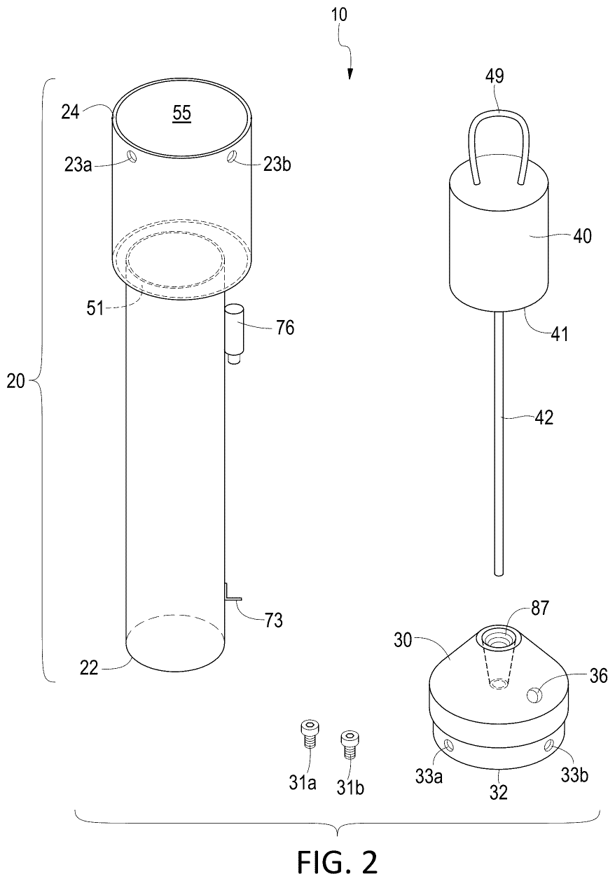

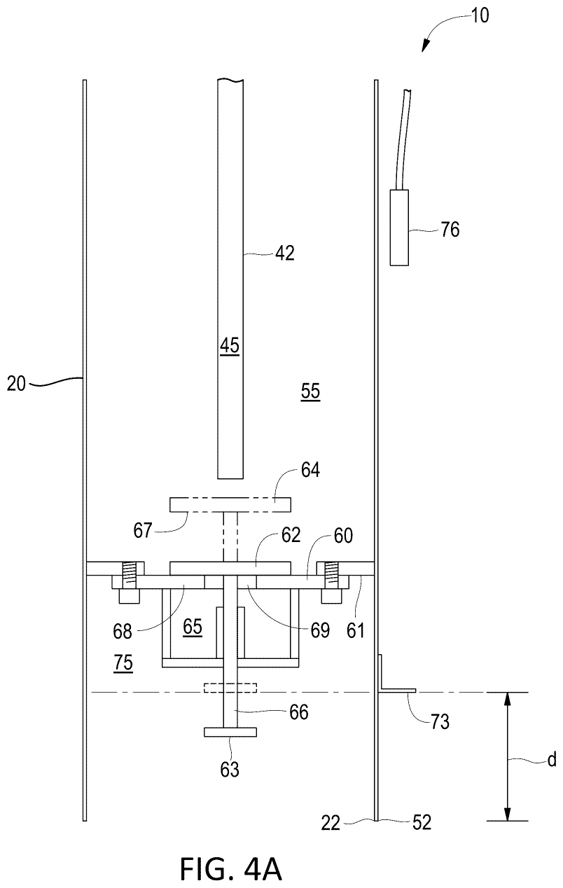

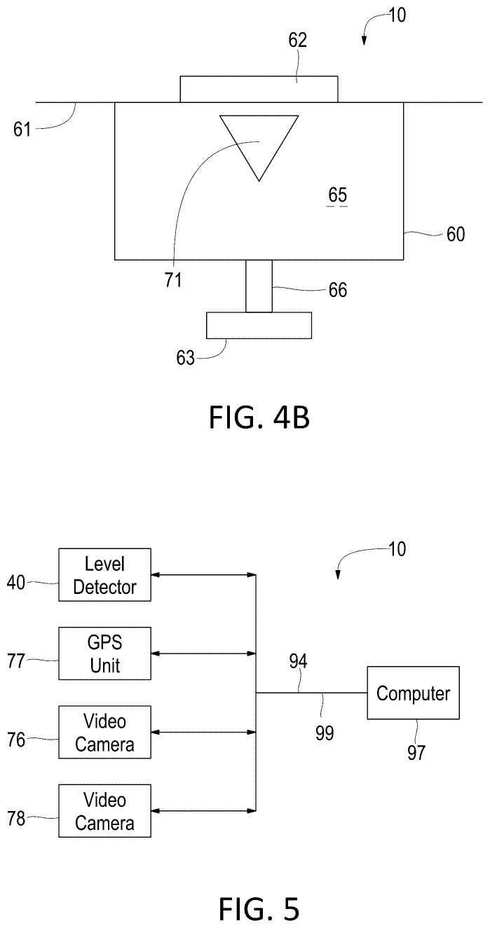

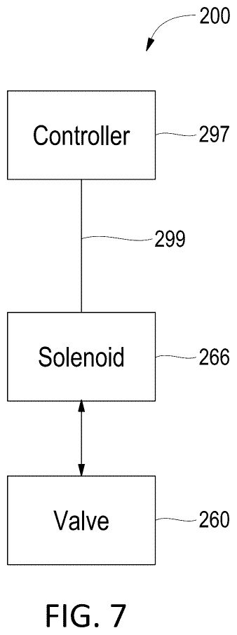

FIG. 1 illustrates exemplary system for falling head infiltrometer testing 10 including infiltrometer 50, with infiltrometer 50 comprised of crown 30 attached to body 20 (also see FIG. 2). Infiltrometer 50 defines infiltrometer passage 55 within, as illustrated. Exemplary system for falling head infiltrometer testing 10 may further include level detector 40 illustrated as placed within infiltrometer passage 55 of infiltrometer 50. Body 20 is cylindrical in shape about axis 101 and linearly aligned with axis 101 and thus with borehole 15, as illustrated. Body 20 defines first body end 22 and second body end 24, and crown 30, which is generally hemispherical in shape, is attached to second body end 24 of body 20 with infiltrometer passage 55 being defined by body 20 in combination with crown 30, as illustrated. First body end 22 and second body end 24 are generally perpendicular to axis 101, and the hemispherical shape of crown 30 is generally centered at axis 101. First body end 22 forms first infiltrometer end 52, and portions of crown 30 form second infiltrometer end 54. Crown 30 may be attached removably to drill stem 88 at coupling 87 proximate second infiltrometer end 54, and drill stem 88 may be manipulated to traverse infiltrometer 50 within borehole 15. Borehole 15 is lined by casing 80 that forms casing passage 85, in this implementation. As illustrated in FIG. 1, infiltrometer 50 lies within casing passage 85 and first infiltrometer end 52 of infiltrometer 50 is inserted into soil 92 to insertion depth d. Following insertion, a valve, such as valve 60, 260, is positioned (see FIGS. 4A, 4B, 7) thereby releasing water 14 from infiltrometer passage 55 into soil 92 thus forming wetted zone 91 within soil 92, as illustrated in FIG. 1. Infiltrometer passage 55 is vented to the atmosphere so that the pressure at water surface 16 is atmospheric and the pressure in water 14 between water surface 16 and soil surface 96 is hydrostatic thus applying positive hydrostatic pressure to soil 92 at soil surface 96. There is no tension in the water column between water surface 16 and soil surface 96.

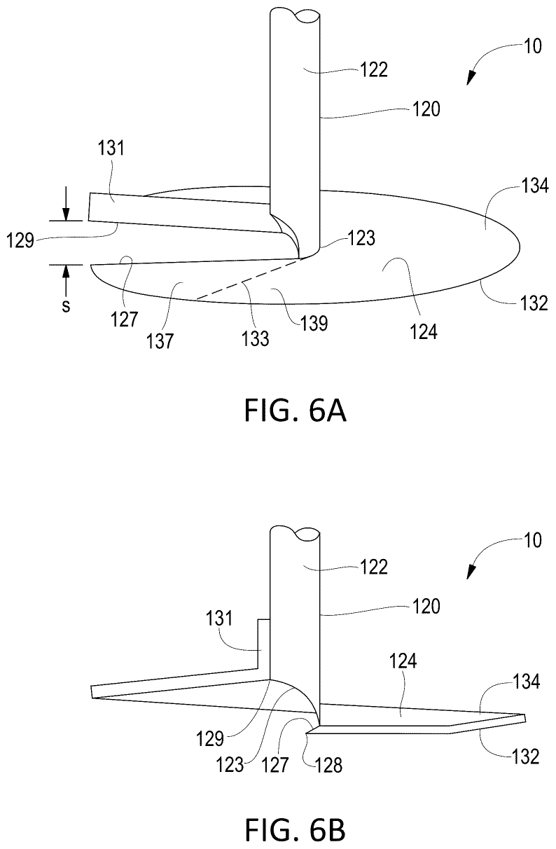

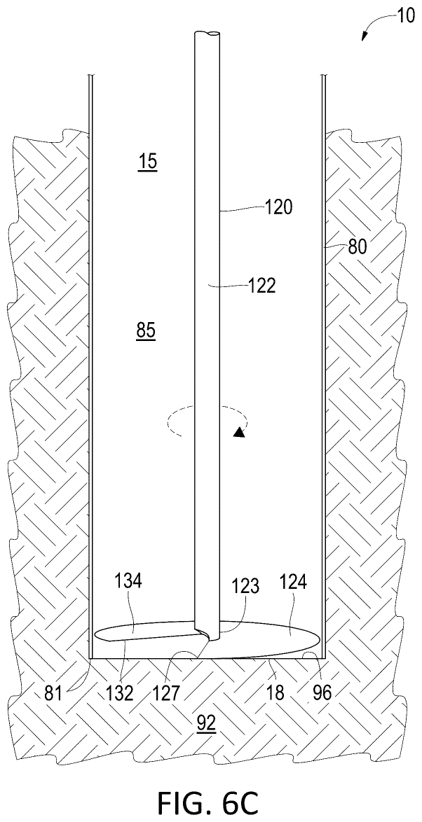

Casing 80 is received within cylindrical borehole 15, which is formed by casing 80, as illustrated, so that infiltrometer 50 is received within borehole 15. Infiltrometer 50 including body 20 and crown 30 may be variously comprised of, for example, steel including stainless steel, aluminum, bronze, hard plastic, in various implementations. As illustrated in FIG. 1, casing end 81, which is open into casing passage 85, is positioned at test elevation y thereby exposing soil surface 96 of soil 92 at test elevation y within casing passage 85 at casing end 81. Soil surface 96 defines borehole bottom 18, as illustrated in FIG. 1 (also see FIG. 6C).

Casing end 81 of casing 80 may be driven into soil 92 to test elevation y with respect to some datum at which soil properties are to be measured in various ways, as would be readily recognized by those of ordinary skill in the art upon study of this disclosure. For example, as illustrated in FIG. 1, casing 80, in this implementation, is configured as a hollow stem auger with spiral flight 89 wrapped externally around casing 80. Note that casing 80 may include one or more flights, in various implementations. A drill rig (not shown) that includes a powerhead and hydraulics may be utilized to drive casing 80 into soil 92, for example. For example, a center plug (not shown) may be provided within casing passage 85 and cutter head (not shown) may be provided at casing end 81 of casing 80 having the hollow stem auger configuration during driving of casing 80 into soil 92. The cutter head cooperates with the drill rig, and the cutter head may have various configurations depending upon soil 92, in such implementations, as would be readily recognized by those of ordinary skill in the art upon study of this disclosure. Upon casing end 81 of casing 80 reaching test elevation y, the center plug and cutter head may be removed to expose soil surface 96 at borehole bottom 18 thereby allowing access to soil surface 96 at borehole bottom 18 through casing 80, as illustrated.

Drill stem 88 may be manipulated exteriorly of casing passage 85 at casing end 83, for example, by the drill rig in order to place infiltrometer 50 within casing passage 85, insert first infiltrometer end 52 of infiltrometer 50 into soil 92 to insertion depth d, and withdraw infiltrometer 50 from casing passage 85. Drill stem 88 is formed, for example, as a bar or pipe capable of carrying both axial compression and axial tension, and may be comprised of steel. Thus, force may be applied axially (e.g., with respect to axis 101) along drill stem 88 to insert first infiltrometer end 52 of infiltrometer 50 into soil 92. The axial force may be applied by hydraulics, in some implementation. In other implementations, drill stem 88 may be manipulated by hand within casing passage 85 including the insertion of first infiltrometer end 52 of infiltrometer 50 into soil 92.

As illustrated in FIG. 1, casing 80 biases against borehole wall 11 of borehole 15 formed within soil 92 to prevent collapse of the borehole 15. While exemplary casing 80 has a hollow stem auger configuration in exemplary system for falling head infiltrometer testing 10 for explanatory purposes, it should be recognized that other soil boring techniques such as, for example, cable tool (percussive) drilling or rotary drilling may be used to form borehole 15 in other implementations. Accordingly, casing 80 should be understood as including any casing as may be provided in conjunction with formation of borehole 15 by, for example, insertion of a hollow stem auger, cable tool drilling, or rotary drilling. For example, in various implementations, casing 80 may be configured without spiral flight 89 (e.g., as a casing pipe). In other implementations, when, for example, soil 92 is sufficiently stable or borehole 15 is shallow, no casing, such as casing 80, may be provided, so that infiltrometer 50 is traversed through borehole 15 without intervening structure, such as casing 80, interposed between infiltrometer 50 and borehole wall 11 of borehole 15. Infiltrometer 50 is in gapped relation with casing 80 so that casing passage 85 is in communication with the ambient atmosphere throughout including exterior of infiltrometer 50 at soil surface 96. In various implementations, borehole 15 may be, for example, at least 4 ft deep. In certain implementations, borehole 15 may be formed using a posthole digger with the resulting borehole being about 3 ft to about 5 ft deep, for example. As would be readily recognized by those of ordinary skill in the art upon study of this disclosure, boreholes formed by drilling may have various depths, for example, 10 ft deep, 20 ft deep, or more. Thus, in various implementations, borehole 15 is generally circular and may have a diameter within a range of 4 in. to 8 in. with a 6 in. diameter being typical. Of course, borehole 15 may have various other diameters in various other implementations, as would be readily recognized by those of ordinary skill in the art upon study of this disclosure.

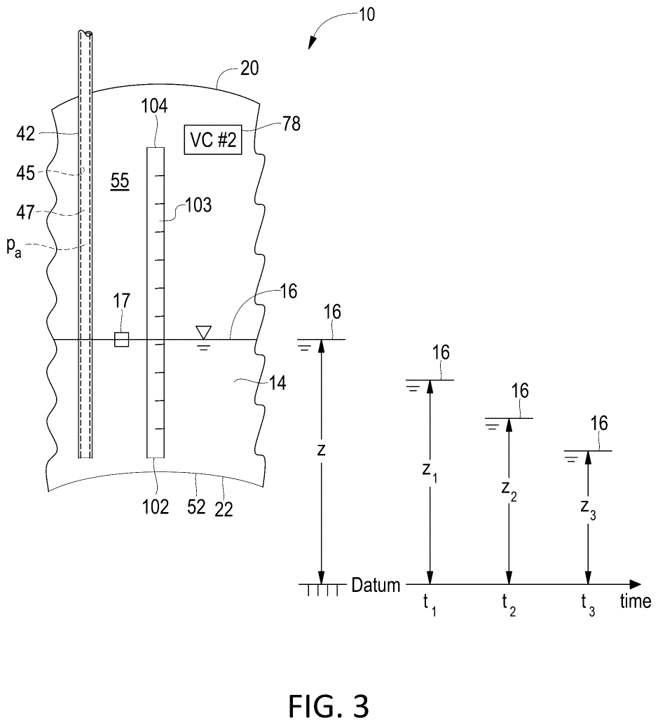

Crown 30 includes port 36 that accesses infiltrometer passage 55 therethrough, in this implementation. Port 36 may allow ventilation of infiltrometer passage 55 to prevent vacuum formation within infiltrometer passage 55 thereby maintaining pressure in water 14 between water surface 16 and soil surface 96 at positive gauge pressure having a hydrostatic pressure distribution. Note that vacuum formation within infiltrometer passage 55 would induce tension in the water column thus interfering with the falling head infiltrometer test. As illustrated, communication pathway 99 passes through port 36 into infiltrometer passage 55 to communicate with level detector 40 and internal video camera 78 (see FIGS. 3, 5), and communication pathway 99 communicates with external video camera 76 affixed externally to infiltrometer 50 (also see FIG. 5).

As illustrated in FIG. 2, crown 30 is separable from body 20 to allow placement of level detector 40 within infiltrometer passage 55 proximate second body end of body 20 as well as removal of level detector 40 from infiltrometer passage 55. Level detector 40 includes handle 49 that allows insertion into or removal of level detector 40 from infiltrometer passage 55. A diameter of cylindrically shaped body 20 is increased proximate second body end 24 to form shelf 51 upon which side 41 of level detector 40 may rest within infiltrometer passage 55. Note shelf 51 is offset slightly to allow passage of communication pathways to external video camera 76 through shelf 51, in this implementation. Also note that the hemispherical shape of crown 30 and the cylindrical shapes of body 20 in this implementation are exemplary, and crown 30 and/or body 20 may assume other geometric shapes in other implementations.

Level detector 40 detects water surface level z of water surface 16 with respect to a reference datum including changes of water surface level z of water surface 16 with respect to time t, in this implementation (also see FIG. 3). As illustrated in FIG. 2, level detector 40 includes tube 42 that extends forth from side 41 of level detector 40 through portions of infiltrometer passage 55 toward first infiltrometer end 52 to measure water surface level z of water surface 16 within infiltrometer passage 55. In this exemplary implementation, level detector 40 measures water surface level z by detecting air pressure pₐ within tube 42. Air pressure pₐ within tube 42 decreases as water surface level z decreases. In various other implementations, level detector 40 may include, for example, a laser, an infrared sensor, SONAR, triangulation, machine vision, or a wave probe (either capacitive or resistive) configured to measure water surface level z. Level detector 40 may communicate data 94 indicative of the water surface level z of water surface 16 as a function of time z (t) including the rate of change of water surface level z of water surface 16 with respect to time (dz/dt) to computer 97 via communication pathway 99, and computer 97 may use data 94 to determine soil properties of soil 92. Computer 97 may determine the soil properties contemporaneously or essentially in real time upon collection of data 94 comprising water surface level as a function of time z (t). Level detector 40 may variously include, for example, a microprocessor, A/D converter, D/A converter, clock, memory, pressure transducer(s), power source, data communication hardware, operatively received software, and such may cooperate variously with communication pathway 99, as would be readily recognized by those of ordinary skill in the art upon study of this disclosure. As would be readily recognized by those of ordinary skill in the art upon study of this disclosure, various power source(s) such as a battery, power connector(s), data connector(s) such as an Ethernet port or a USB port, electrical transformers, power inverters, user interface(s), switch(es), electrical pathway(s), and so forth may be included in level detector 40, infiltrometer 50, or otherwise disposed about system for falling head infiltrometer testing 10, and such may cooperate variously with communication pathway 99, in various implementations.

In this implementation, crown 30 is removably attachable to body 20 by insertion of flange 32 of crown 30 into body 20 at second body end 24 and then securement of crown 30 to body 20 using fasteners, such as fasteners 31a, 31b, as illustrated in FIG. 2. Fasteners, such as fasteners 31a, 31b, pass through holes, such as holes 23a, 23b, provided in body 20, to threadedly engage holes, such as holes 33a, 33b, provided in crown 30, in this exemplary implementation. Fasteners, such as fasteners 31a, 31b, may include, for example, various bolts, screws, nuts, pins, threaded male and/or female members, in various implementations. Exemplary coupling 87 of crown 30 is formed with an AWJ thread allowing crown 30 to be threadedly coupled with drill stem 88, in this exemplary implementation. Drill stem 88 may be coupled mechanically to infiltrometer 50 in various other ways using, for example, various other threads or coupling devices, in various other implementations.

As illustrated in FIG. 3, tube 42 of level detector 40 extends forth from side 41 of level detector 40 downward through at least portions of infiltrometer passage 55 toward first infiltrometer end 52. Tube 40 forms tube passage 45 with water surface 16 at water surface level z within tube passage 45 and within infiltrometer passage 55 (assuming negligible surface tension within tube passage 45), as illustrated in FIG. 3. Portions of tube passage 45 are occupied by air 47 at pressure pₐ, and air pressure pₐ changes as water surface level z changes, in this implementation. For example, the air pressure pₐ within tube passage 45 decreases in correspondence as water surface level z decreases as water 14 is released from infiltrometer passage 55 by valve 60 (see FIGS. 4A, 4B) to infiltrate into soil 92. Using pressure pₐ, level detector 40 may then communicate data 94 indicative of water surface level as a function of time z (t) to computer 97 via communication pathway 99, in this implementation. For example, data 94 may be indicative of a plurality of water surface levels z₁, z₂, z₃ . . . at a corresponding plurality of times t₁, t₂, t₃ . . . , as illustrated in FIG. 3.

In exemplary system for falling head infiltrometer testing 10, internal video camera 78 (shown schematically in FIG. 3) is disposed within infiltrometer passage 55 to allow observation of water surface 16. As illustrated, float 17 floats upon water surface 16 to facilitate observation of water surface 16 by internal video camera 78. Internal video camera 78 captures visual information in digital form, and internal video camera 78 may include a light source (not shown) in order to observe water surface 16. Scale 103 disposed within infiltrometer passage 55 is also observable by internal video camera 78, in this implementation. Accordingly, water surface level as a function of time z (t) within infiltrometer passage 55 may be observed by internal video camera 78 using scale 103 including water surface levels z₁, z₂, z₃ . . . at a corresponding plurality of times t₁, t₂, t₃ . . . , as illustrated in FIG. 3. Data 94 may include visual information in digital form as observed by internal video camera 78 communicated from internal video camera 78 to computer 97 via communication pathway 99, and the data 94 may be stored in non-transient form. Accordingly, data 94 may include water surface levels z₁, z₂, z₃ . . . at a corresponding plurality of times t₁, t₂, t₃ . . . as observed by internal video camera 78 using scale 103.

As illustrated in FIGS. 4A, 4B, valve 60 is mounted within infiltrometer passage 55 proximate first infiltrometer end 52 by valve mount 61. As illustrated, valve 60 includes valve piston 66 slidably mounted in relation with valve seat 68. Valve seat 68 forms inflow port 69 for fluid communication therethrough into valve chamber 65. As illustrated in FIG. 4B, outflow ports, such as outflow port 71, are disposed circumferentially around valve chamber 65 of valve 60 for fluid communication out of valve chamber 65. The circumferential disposition of the outflow ports may prevent scour of soil surface 96.

Valve 60 is positionable by motion of valve piston 66 between a closed position 62 (illustrated in FIG. 4A in solid line) that retains water 14 within infiltrometer passage 55 and open position 64 (illustrated in FIG. 4A in phantom) that releases water from infiltrometer passage 55 to infiltrate into soil 92. With infiltrometer 50 oriented vertically, valve 60 is held in closed position 62 by gravity that holds piston face 67 in contact with valve seat 68 blocking inflow port 69 thereby blocking water flow through valve 60. Contact of piston face 63 of valve 60 with soil surface 96 of soil 92 as first infiltrometer end 52 of infiltrometer 50 is inserted into soil 92 to insertion depth d positions valve 60 from closed position 62 to open position 64 by lifting valve piston 66 thereby placing piston face 67 in gapped relation with valve seat 68 and allowing water flow through inflow port 69 of valve seat 68 and, thus, through valve 60 thereby generally filling region 75 between valve mount 61 and soil surface 96. With valve 60 in open position 64, water 14 is released from infiltrometer passage 55 by communication through inflow port 69, though valve chamber 65, through outflow ports such as outflow port 71 disposed around valve 60 (see FIG. 4B) and thence onto soil surface 96 to infiltrate into soil 92. When valve 60 is in open position 64 during an infiltration test, the pressure distribution within infiltrometer passage 55 between water surface 16 and soil surface 96 (all of which is occupied by water) is hydrostatic with water surface 16 being at ambient atmospheric pressure because air flow may be communicated between the atmosphere and water surface 16 through port 36 into infiltrometer passage 55.

As illustrated in FIG. 4A, soil stop 73, which may be formed as a ½ inch angle iron in certain implementations, is disposed externally on infiltrometer 50 at insertion depth d above first infiltrometer end 52 of infiltrometer 50 to limit insertion of first infiltrometer end 52 of infiltrometer 50 into soil 92 to insertion depth d or at least indicate insertion to insertion depth d. In some implementations, soil stop 73 may physically limit the insertion of first infiltrometer end 52 of infiltrometer 50 into soil 92 through soil surface 96 to insertion depth d. External video camera 76 may be used to guide infiltrometer 50 through borehole 15 including insertion of first infiltrometer end 52 into soil 92. For example, soil stop 73 may be observed using external video camera 76, and insertion of first infiltrometer end 52 of infiltrometer 50 into soil 92 may be halted when soil stop 73 is observed touching soil surface 96 using external video camera 76. External video camera 76 may include a light source (not shown) to illuminate borehole 15 including soil stop 73 and soil surface 96. External video camera 76 may communicate data 94 comprising visual information captured by external video camera 76 with computer 97 for example via communication pathway 99.

As illustrated in FIG. 5, exemplary system for falling head infiltrometer testing 10 includes Global Positioning System (GPS) unit 77 that communicates data 94 indicative, for example, of the GPS location of the test location at which the water surface levels z₁, z₂, z₃ . . . at corresponding times t₁, t₂, t₃ . . . are detected by level detector 40. GPS unit 77 may be a GPS chip or other such device capable of receiving information, for example, from GPS satellites and calculating GPS location using that information. For example, level detector 40 may include GPS unit 77, in some implementations. Computer 97 may include GPS unit 77, in other implementations. It should be noted that computer 97 may assume various locations and configurations, in various implementations. That is, the illustration of computer 97 in the Figures is representational provided only for explanatory purposes and is not limiting.

As illustrated in FIG. 5, level detector 40, GPS unit 77, external video camera 76, and internal video camera 78 communicate data 94 with computer 97 via communication pathway 99. Computer 97 may variously interact with level detector 40, GPS unit 77, external video camera 76, and internal video camera 78 to control level detector 40, GPS unit 77, external video camera 76, and internal video camera 78, so that data 94 may include various control instructions communicated from computer 97. Communication pathway 99 may include various wired and wireless communications pathways and combinations thereof. For example, communication pathway 99 may variously include the Internet, local area networks (LAN), cell phone networks (e.g., 4G, 5G), text messaging networks (such as MMS or SMS networks), wide area networks (WAN), and combinations thereof. Communication pathway 99 may be wired (e.g., optical, electromagnetic), wireless (e.g., infra-red (IR), electromagnetic), or a combination of wired and wireless, and communication pathway 99 may conform, at least in part, to various standards, (e.g., Bluetooth®, ANT, ZigBee, FDDI, ARCNET IEEE 802.11, IEEE 802.20, IEEE 802.3, IEEE 1394-10395, USB). Communication pathway 99 may variously communicate electrical power, analog signals, and data 94. Communication pathway 99 may include, for example, processors, data storage devices, input/output devices, computers, servers, routers, amplifiers, wireless transmitters, wireless receivers, optical devices, A/D converters, D/A converters, power communication devices, virtualized resources, and so forth, as would be readily recognized by those of ordinary skill in the art upon study of this disclosure.

System for falling head infiltrometer testing 10 may include exemplary tool 120 illustrated in FIGS. 6A, 6B, 6C. As illustrated in FIGS. 6A, 6B, 6C, tool 120 includes shaft 122 with blade 124 affixed in spiral disposition around shaft 122 proximate shaft end 123 of shaft 122. First blade portion 137 of blade 124 is generally flat, e.g., generally perpendicular to shaft 122. Second blade portion 139 of blade 124 is then disposed spirally around shaft starting at dividing line 133 in FIG. 6A between first blade portion 137 and second blade portion 139. That is, blade 124 starts to bend from flat first blade portion 137 into spirally curved second blade portion 139 proximate dividing line 133. As illustrated, blade 124 makes about one 360° rotation around shaft 122 between blade ends 127, 129. Shaft 122 may be segmented to be extendable to allow insertion of blade 124 through an entirety of casing passage 85 from casing end 83 to soil surface 96 at borehole bottom 18 at casing end 81 and allow manipulation of blade 124 at soil surface 96 by manipulation of shaft 122 exteriorly of casing passage 85 at casing end 83. Blade 124 may be generally sized to have a diameter generally commensurate with a diameter of casing passage 85 or borehole 15. For example, blade 124 may be about 6 in. (15 cm) in diameter.

Blade end 127 of blade 124, which is disposed nearer shaft end 123, is beveled to form edge 128, and blade end 129 of blade 124, which is disposed further from shaft end 123, is formed as flange 131. Edge 128 may be beveled at about 30° with respect to surface 132. Blade end 129 is offset from blade end 127 by length s, as illustrated, where length s may be, for example, about 1 in. (2.5 cm). Note that shaft end 123 is curved to pass between blade ends 127, 127 as illustrated in FIGS. 6A, 6B. Blade 124 may be formed of 0.1875 in. thick mild steel plate, for example, and shaft 122 may be formed of 0.875 in. diameter steel rod.

When tool 120 is inserted through borehole 15 with surface 132 of blade 124 contacting soil surface 96 and shaft 122 is rotated clockwise, as illustrated in FIG. 6C, edge 128 scoops soil 92 proximate soil surface 96 onto surface 134 of blade 124, and flange 131 then retains the soil 92 on surface 134. Soil 92 on surface 134 of blade 124 may then be removed by withdrawal of tool 120 from borehole 15. Thus, tool 120 may be used, for example, to excavate soil 92 in order to expose soil surface 96 having an undisturbed condition for a more accurate infiltration test, to remove debris from soil surface 96 such as may be accumulated during placement of casing 80 or boring of borehole 15, and to obtain soil 92 from proximate soil surface 96 for measurement of certain soil properties thereof such as moisture content that may be required for the determination of soil properties by infiltrometer testing.

Portions of another system for falling head infiltrometer testing 200 are illustrated in FIG. 7. As illustrated, solenoid 266 cooperates with valve 260 to position valve 260 to retain water within an infiltrometer passage, such as infiltrometer passage 55, or to release water from the infiltrometer passage into soil, such as soil 92. Controller 297 communicates with solenoid 266 via communication pathway 299 to effectuate valve 260, in this implementation. Controller 297 may be configured as a computer, such as computer 97, in various implementations, and controller 297 may be positioned external of a casing passage, such as casing passage 85.

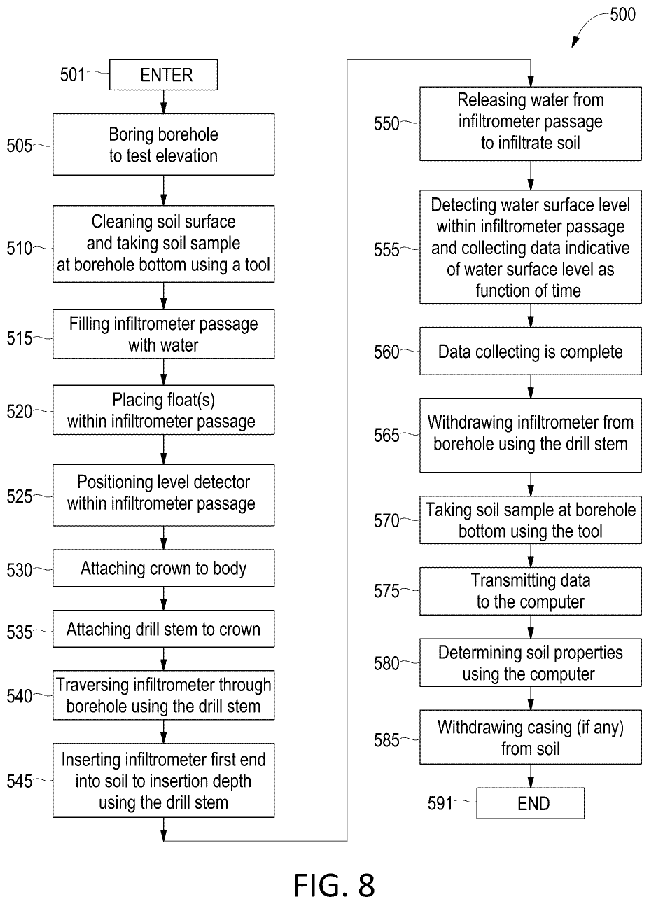

Exemplary operations of a system for falling head infiltrometer testing, such as system for falling head infiltrometer testing 10, 200, may generally follow exemplary method 500 illustrated in FIG. 8. Note that method 500 including the steps therein is exemplary and provided for explanatory purposes. Thus, it should be recognized, for example, that the steps of method 500 may be combined, performed in other orders, or omitted, in various other implementations.

Exemplary method 500 is entered at step 501. At step 505, a borehole, such as borehole 15 is bored into a soil, such as soil 92, to a test elevation, such as test elevation y, which lies below grade thereby exposing a soil surface, such as soil surface 96, of the soil at the test elevation. The borehole may be lined with a casing forming a casing passage, such as casing 80 forming casing passage 85, and a casing end, such as casing end 81, of the casing is positioned at the test elevation open to expose a soil surface, such as soil surface 96, at the borehole bottom, such as borehole bottom 18. The borehole may be unlined in certain implementations.

At step 510, a tool, such as tool 120, is inserted into the borehole and rotated to remove debris from the soil surface at the borehole bottom accumulated during boring of the borehole and/or placement of the casing (if any) to excavate the soil in order to expose the soil surface in an undisturbed state, and to obtain soil from proximate the soil surface for measurement of the moisture content thereof prior to starting the infiltrometer test at step 550.

At step 515, a portion of an infiltrometer passage within a body, such as a portion of infiltrometer passage 55 within body 20, is filled at least in part with water, such as water 14, through a second body end, such as second body end 24, of the body. The portion of the infiltrometer passage may be filled with water approximately up to a scale end of a scale, such as scale end 104 of scale 103. A valve, such as valve 60, 260, is in a closed position, such as closed position 62, to retain the water within the infiltrometer passage during step 515.

At step 520, one or more floats, such as float 17, are placed into the infiltrometer passage to identify visually a water surface, such as water surface 16, within the infiltrometer passage. An internal video camera, such as internal video camera 78, may observe a water surface level z within the infiltrometer passage with respect to the scale with the aid of the one or more floats at step 555.

At step 525, a level detector, such as level detector 40, is then positioned within the infiltrometer passage, for example, upon a shelf, such as shelf 51, formed within the infiltrometer passage.

At step 530, with the infiltrometer passage containing water and level detector positioned therein, a crown, such as crown 30, is then attached to the second body end of the body thereby forming an infiltrometer, such as infiltrometer 50. At least portions of one or more communication pathways, such as communication pathway 99, configured as cable(s) may be passed through a port, such as port 36, formed in the crown for communication, for example, with variously the internal video camera, the level detector, and a solenoid, such as solenoid 266, in conjunction with step 530. Fasteners, such as fasteners 31a, 31b, may be used to secure removably the crown to the body.

At step 535, a drill stem, such as drill stem 88, is attached to the crown of the infiltrometer using a coupling, such as coupling 87, the crown having been attached to the body at step 530.

At step 540, the drill stem is then manipulated to insert the infiltrometer into the borehole and traverse the infiltrometer through the borehole. Water is retained within the infiltrometer passage by the valve during step 540. In some implementations, the infiltrometer is inserted into the casing passage at a casing end, such as casing end 83. Note that the casing has been driven into the soil to the test elevation per step 505.

At step 545, the first infiltrometer end is inserted into the soil through the soil surface at the borehole bottom to an insertion depth, such as insertion depth d, below the soil surface using the drill stem. For example, an external video camera, such as external video camera 76, attached to the infiltrometer may be used to observe a soil stop, such as soil stop 73, disposed exteriorly to the infiltrometer and the soil surface during insertion with insertion of the first infiltrometer end to the insertion depth being indicated by the soil stop contacting the soil surface. The external video camera may communicate by the communication pathway. Note that compression is applied to the drill stem in order to forcibly compressionally insert the first infiltrometer end into the soil to the insertion depth. Accordingly, a cable, chain, rope, or suchlike that cannot transmit a compressive force may not be utilizable in lieu of the drill stem.

At step 550, the water is released from the infiltrometer passage to infiltrate soil by positioning of the valve from the closed position to an open position, such as open position 64. In some implementations, for example, contact of the valve with the soil by insertion of the first infiltrometer end to the insertion depth positions the valve from the closed position to the open position. In other implementations, for example, a solenoid may be signaled to position the valve from the closed position to the open position.

At step 555, water surface level as a function of time z (t) within infiltrometer passage is detected as the water infiltrates into the soil. The level detector may detect water surface level as a function of time z (t) as per step 555 and accumulate as data, such as data 94. At step 555, the internal video camera may observe the water surface level z within the infiltrometer passage with respect to the scale thereby observing water surface levels z₁, z₂, z₃ . . . at a corresponding plurality of times t₁, t₂, t₃ . . . . The data may include visual information in digital form as observed by the internal video camera, and the data may be communicated from the internal video camera to the computer via the communication pathway. Accordingly, the data may include water surface levels z₁, z₂, z₃ . . . at a corresponding plurality of times t₁, t₂, t₃ . . . as observed by the internal video camera using the scale. The water surface levels z₁, z₂, z₃ . . . at a corresponding plurality of times t₁, t₂, t₃ . . . as observed by the internal video camera may then be used to determine soil properties of the soil at the test elevation y. Thus, at step 555, the water surface level as function of time z (t) may be determined using the level detector, using the internal video camera with the scale, or both the level detector and the internal video camera and the scale. The data is indicative of water surface level as function of time z (t), and the data may be stored in non-transient form, for example, by the level detector or by the computer. The data may be indicative of a rate of change of the water surface level of the water surface with respect to time (dz/dt).

At step 560, the collection of the data comprising water surface level as function of time z (t) is now complete. For example, the water surface falling below a scale end, such as scale end 102, of the scale may signify the end of collection of water surface level as function of time z (t) data.

At step 565, the infiltrometer is withdrawn from the borehole using the drill stem, the infiltration test of the soil at the test elevation having been concluded.

At step 570, the tool is inserted into the borehole and rotated to obtain soil from proximate the soil surface at the borehole bottom, for example, for measurement of the moisture content thereof.

At step 575, the data is transmitted to the computer. For example, the level detector may be connected to the computer via the communication pathway for data transmission of the data from the level detector to the computer. In other implementations, the data may be transmitted by the communication pathway in real time during the infiltration test. The data may include soil moisture of soil samples obtained using the tool at step 510 and at step 570.

At step 580, the computer determines the soil properties of the soil at the test elevation by calculations using the data. The computer may record the corresponding GPS location, and the computer may associate portions of the data indicative of the water surface level as function of time z (t), the soil properties determined from the water surface level as function of time z (t), soil moistures, and the GPS location. In certain implementations, the computer may determine the GPS location of the computer, the computer being proximate to the test location at which the data is obtained. The computer may aggregate data indicative of the water surface level z as a function of time and corresponding soil properties at a plurality of GPS locations and/or test elevations thereby mapping soil properties within some geographic region. The computer 97 may communicate with the level detector via the communication pathway, for example, to control, at least in part, the detection of the water surface level z as a function of time such as, for example, the times t₁, t₂, t₃ . . . at which water surface levels z₁, z₂, z₃ . . . are measured.

At step 585, the casing, if any, is withdrawn from the borehole and the borehole may then be refilled with soil. Alternatively, at step 585, the borehole may be bored deeper below grade to another test elevation y and steps 510 to 580 repeated. Method 500 terminates at step 591.

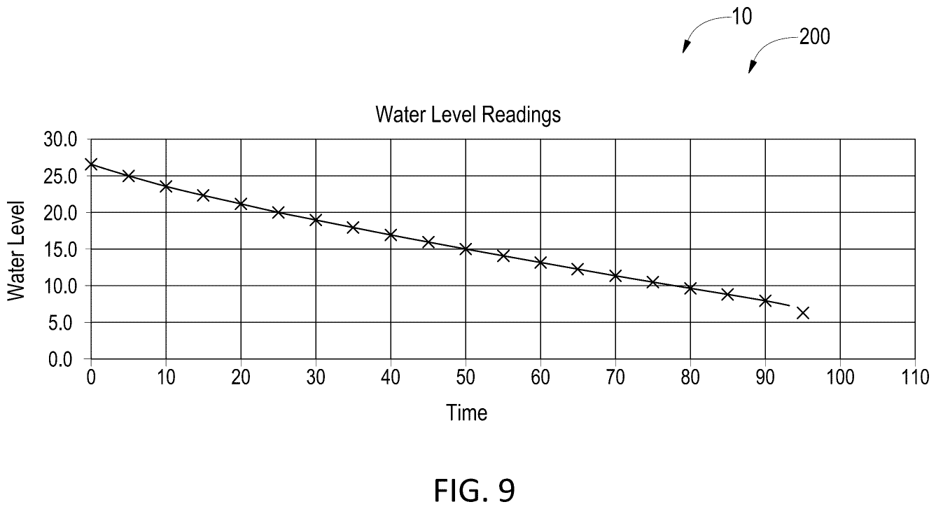

FIG. 9 illustrates graphically exemplary water surface levels z₁, z₂, z₃ . . . at a corresponding plurality of times t₁, t₂, t₃ . . . as collected by the level detector, by observation using the internal video camera, or both by the level detector and observation using the internal video camera of the system for falling head infiltrometer testing. The exemplary water surface level as a function of time z (t) of FIG. 9 result from a falling head infiltrometer test and may be utilized by the computer to determine the soil properties of the soil at the test elevation. Note that water surface levels detected prior to the initiation of the infiltrometer test (constant) and after conclusion of the infiltrometer test, if any, have been removed from exemplary FIG. 9. The computer may remove automatically such extraneous values during determination of the soil properties of the soil by calculation at step 580.

The foregoing discussion along with the Figures discloses and describes various exemplary implementations. These implementations are not meant to limit the scope of coverage, but, instead, to assist in understanding the context of the language used in this specification and in the claims. The Abstract is presented to meet requirements of 37 C.F.R. § 1.72 (b) only. Accordingly, the Abstract is not intended to identify key elements of the apparatus and methods disclosed herein or to delineate the scope thereof. Upon study of this disclosure and the exemplary implementations herein, one of ordinary skill in the art may readily recognize that various changes, modifications and variations can be made thereto without departing from the spirit and scope of the inventions as defined in the following claims.

The invention claimed is:

1. A system for falling head infiltrometer testing, comprising:
an infiltrometer defining an infiltrometer passage within and having a first infiltrometer end and a second infiltrometer end, the infiltrometer being configured for traversal of a borehole by coupling of the second infiltrometer end to a drill stem in order to insert the first infiltrometer end into a soil at a borehole bottom of the borehole to an insertion depth;
a valve disposed proximate the first infiltrometer end of the infiltrometer; and
wherein the valve is positionable between a first valve position that retains water within the infiltrometer passage and a second valve position that releases water from the infiltrometer passage.

2. The system of claim 1, wherein the valve is positionable from the first valve position to the second valve position by contact of the valve with a soil surface of the soil at the borehole bottom of the borehole upon insertion of the first infiltrometer end of the infiltrometer into the soil.

3. The system of claim 1, further comprising:
a level detector removably placeable within the infiltrometer passage, the level detector operable to detect a water surface level of a water surface within the infiltrometer passage as a function of time.

4. The system of claim 1, further comprising:
an external video camera attached externally to the infiltrometer, the external video camera configured to view insertion of the first infiltrometer end of the infiltrometer into a soil surface of a soil at a borehole bottom of the borehole to an insertion depth.

5. The system of claim 4, further comprising:
a marker affixed externally to the infiltrometer viewable by the external video camera, the marker indicative of the first infiltrometer end being inserted into the soil to the insertion depth.

6. The system of claim 1, further comprising:
an internal video camera attached to the infiltrometer within the infiltrometer passage, the internal video camera configured to view the water surface level of the water surface within the infiltrometer passage.

7. The system of claim 1, further comprising:
a tool to remove soil from the borehole bottom of the borehole, the tool comprising:
a shaft configurable to extend at least a length of the borehole;
a blade affixed in spiral shaped disposition to the shaft proximate a shaft end of the shaft and having a diameter generally commensurate with a diameter of a borehole passage;
a blade end of a blade portion disposed nearest the shaft end formed as an edge to scoop soil proximate a soil surface onto a surface of the blade by rotation of the blade; and
a blade end of a blade portion disposed furthest from the shaft end formed as a flange to retain soil upon the surface of the blade.

8. The system of claim 1, further comprising:
a communication pathway to communicate data between a device and a computer, the device selected from a group consisting of a level detector, an internal video camera, and an external video camera.

9. A system for falling head infiltrometer testing, comprising:
an infiltrometer defining an infiltrometer passage within and having a first infiltrometer end and a second infiltrometer end, the infiltrometer passage containing water, the first infiltrometer end being inserted into a soil surface of a soil at a borehole bottom of a borehole to an insertion depth within the soil;
a drill stem coupled to the second infiltrometer end of the infiltrometer to traverse the infiltrometer within the borehole;
a valve disposed proximate the first infiltrometer end of the infiltrometer; and
wherein the valve is positioned between a first valve position that retains the water within the infiltrometer passage during traversal of the infiltrometer within the borehole and a second valve position that releases the water from the infiltrometer passage into the soil surface of the soil at the borehole bottom of the borehole upon insertion of the first infiltrometer end into the soil to the insertion depth.

10. The system of claim 9, further comprising:
a level detector placed within the infiltrometer passage, the level detector operable to detect a water surface level of a water surface of water within the infiltrometer passage as a function of time and operable to generate data indicative of the water surface level as the function of time.

11. The system of claim 9, further comprising:
an internal video camera attached to the infiltrometer within the infiltrometer passage, the internal video camera configured to view the water surface within the infiltrometer passage.

12. The system of claim 9, further comprising:
an external video camera attached externally to the infiltrometer, the external video camera configured to view insertion of the infiltrometer first end of the infiltrometer into the soil to the insertion depth as indicated by a marker.

13. The system of claim 9, further comprising:
a tool to remove soil from the borehole bottom of the borehole, the tool comprising:
a shaft configurable to extend at least a length of the borehole;
a blade affixed in spiral shaped disposition to the shaft proximate a shaft end of the shaft and having a diameter generally commensurate with a diameter of a borehole passage;
a blade end of the blade disposed nearest the shaft end formed as an edge to scoop soil proximate a soil surface onto a surface of the blade by rotation of the blade; and
a blade end of the blade disposed furthest from the shaft end formed as a flange to retain soil upon the surface of the blade.

14. The system of claim 9, further comprising:
a casing received within the borehole in biased engagement with a borehole wall of the borehole, the casing defining a casing passage, the infiltrometer disposed within the casing passage.

15. The system of claim 14, wherein the casing is configured as a hollow stem auger.

16. The system of claim 9, further comprising:
a communication pathway to communicate data between a device and a computer set apart from the borehole, the device selected from a group consisting of a level detector, an internal video camera, and an external video camera.

17. The system of claim 16, wherein data communicated via the communication pathway is indicative of a plurality of water surface levels at a corresponding plurality of times.

18. The system of claim 17, wherein the computer is configured to use the plurality of water surface levels at a corresponding plurality of times to determine a soil property selected from a group consisting of hydraulic conductivity, porosity, sorptivity, and intrinsic permeability.

19. A method of measuring soil properties, comprising the steps of:
forming a borehole with a borehole bottom at a test elevation thereby exposing a soil surface of a soil at the borehole bottom of the borehole;
inserting an infiltrometer first end of an infiltrometer into the soil to an insertion depth at the borehole bottom of the borehole using a drill stem coupled to a second infiltrometer end of the infiltrometer, the infiltrometer containing water within an infiltrometer passage;
releasing the water from the infiltrometer passage into the soil surface of the soil at the borehole bottom of the borehole by positioning a valve from a first valve position retaining the water within the infiltrometer passage to a second valve position, the infiltrometer first end having been inserted into the soil to the insertion depth; and
collecting data indicative of changes of a water surface level of water within the infiltrometer passage as a function of time following the step of releasing the water from the infiltrometer passage into the soil surface of the soil at the borehole bottom of the borehole.

20. The method of claim 19, further comprising the step of:
determining a soil property selected from a group consisting of hydraulic conductivity, porosity, sorptivity, and intrinsic permeability by a computer using the data.
